(19) United States
(12) Patent Application Publication
(10) Pub. No.: US 2021/0315803 A1
(43) Pub. Date: Oct. 14, 2021

(54) SUSTAINED-RELEASE INJECTABLE ANTIBIOTICAL FORMULATION

(71) Applicant: YISSUM RESEARCH DEVELOPMENT COMPANY OF THE HEBREW UNIVERSITY OF JERUSALEM LTD., Jerusalem (IL)

(72) Inventors: Michael FRIEDMAN, Jerusalem (IL); David KIRMAYER, Maale Adumim (IL); Zakhar NUDELMAN, Netanya (IL); Amnon HOFFMAN, Jerusalem (IL); Eran LAVY, Kibbutz Revadim (IL); Ayala BAR-HAI, Nof Ayalon (IL); Irith GATI, Mevasseret Zion (IL)

(73) Assignee: YISSUM RESEARCH DEVELOPMENT COMPANY OF THE HEBREW UNIVERSITY OF JERUSALEM LTD., Jerusalem (IL)

(21) Appl. No.: 17/273,828

(22) PCT Filed: Sep. 5, 2019
(86) PCT No.: PCT/IL2019/050998

§ 371 Date: Mar. 5, 2021

Related U.S. Application Data
(60) Provisional application No. 62/727,574, filed on Sep. 6, 2018.

(51) Int. Cl. A61K 9/00; A61K 31/165; A61K 47/34; A61K 47/38; A61K 47/22; A61K 47/20; A61K 47/10

(52) U.S. Cl. CPC A61K 9/0019; A61K 31/165; A61K 47/34; A61K 47/10; A61K 47/22; A61K 47/20; A61K 47/38

(57) ABSTRACT

Provided herein are compositions of injectable antibiotics for veterinary use. The compositions are characterized by forming a gel at animal physiological temperature, said gel being characterized by a stable and repeatable release profile of the antibiotic. The compositions comprise high loading of drug in poloxamer solutions with addition of a co-solvent, and preferably with an addition of a cellulose derivative at least partially soluble in organic solvents. Methods of treatment of veterinary infections are also provided.

### FIELD AND BACKGROUND OF THE INVENTION

The present invention relates to a sustained-release formulation, and more specifically, to a sustained-release formulation which is suitable for poorly soluble antibiotics, for veterinary use.

Oral administration of medications which is considered as the preferred route in medicine, is, for obvious reasons, often unfeasible in veterinary medicine, especially when large domestic animals are concerned. For similar reasons, administration of medication which requires multiple dosing is often prove difficult or even impractical.

Sustained release of a drug following parenteral administration is generally preferable to oral administration in veterinary medicine and allows the treatment of large domestic animals (such as cattle) as well as pets and other animals. Reducing the dosing frequency is known to improve patient safety, reduce the incidence of injection site complications and improve compliance with drug protocols. Sustained release formulations mitigate the bolus effect at the time of injection, and thus have a salutary influence on drug side effects. For certain prophylactic uses and treatments, one-time administration or infrequent administration has become a standard procedure. For example, monthly administration is available in most heartworm preventatives such as Heartguard®, Sentinel® and Interceptor medications. Controlled release parenteral formulations may be in the form of liquids, in situ forming solids and solids [Medlicott et al., Advanced Drug Delivery Reviews 2004, 56:1345-1365]. Best-selling parenteral controlled release products include Posilac® milk enhancer (a liquid suspension), Micotil® antibiotic (a liquid solution), Nuflor® antibiotic (a liquid solution) and Revalor® growth enhancer (a solid implant).

In recent years studies involving the use of poloxamers in sustained release formulations have been reported. Poloxamers are nonionic triblock copolymers which consist of blocks of relatively hydrophilic poly(ethylene oxide) (PEO) and relatively hydrophobic poly(propylene oxide) (PPO) arranged in A-B-A tri-block structure: PEO-PPO-PEO. Poloxamer aqueous gels are described, for example, in U.S. Pat. No. 3,740,421. Poloxamers are used as emulsifying agents for intravenous fat emulsions, as solubilising agents to maintain clarity in elixirs and syrups, and as wetting agents for antibacterials. They may also be used in ointment or suppository bases and as tablet binders or coaters [Sweetman (Ed.), Martindale: The Complete Drug Reference, London: Pharmaceutical Press]. The hydrophobic-lipophilic balance (HLB) of a poloxamer may be characterized by the numbers of ethylene oxide and propylene oxide units in the copolymer. Due to their amphiphilic nature, poloxamer copolymers display surfactant properties, including an ability to interact with hydrophobic surfaces and biological membranes. In aqueous solutions at concentrations above the critical micelle concentration (CMC) these copolymers self-assemble into micelles. The diameters of poloxamer micelles usually vary from approximately 10 nm to 100 nm. The core of the micelles consists of hydrophobic PPO blocks that are separated from the aqueous exterior by a hydrated shell of PEO blocks. The core is capable of incorporating various therapeutic or diagnostic reagents [Bartrakova & Kabanov, Journal of Controlled Release 2008, 130:98-106]. Poloxamers are generically designated with the letter P (for “poloxamer”) followed by three digits. The first two digits multiplied by 100 give the approximate molecular mass of the PPO core, and the last digit multiplied by 10 gives the percentage of PEO. For example, P407 is a poloxamer with a PPO molecular mass of 4,000 Da, and a 70% PEO content. According to an additional designation system (used, for example, in association with Pluronic® and Lutrol® tradenames), the copolymer is designated with a letter which defines its physical form at room temperature, L for liquid, P for paste, F for flake (solid), followed by two or three digits. The first digit (or first two digits in a three-digit number) multiplied by 300, indicates the approximate molecular weight of the hydrophobic block, and the last digit multiplied by 10 gives the percentage of polyethylene oxide (PEO). For example, L61 is a liquid poloxamer with a PPO molecular mass of 1,800 Da, and a 10% PEO content, which would be designated as P181 according to the designation system described above.

U.S. Patent Application No. 20090214685 describes a thermoplastic pharmaceutical composition comprising botulinum toxin and a biocompatible poloxamer. The pharmaceutical composition can be administered as a liquid, and gels after administration into a sustained release drug delivery system from which the botulinum toxin is released over a multi-day period. U.S. Pat. No. 7,008,628 describes a pharmaceutical composition which comprises a linear block copolymer such as a poloxamer, end-modified by a bioadhesive polymer such as polyacrylic acid. The polymer is capable of aggregating in response to an increase in temperature. U.S. Pat. No. 7,250,177 describes gel-forming poloxamers modified with a crosslinkable group such as acrylate, which can be crosslinked to form a thermosensitive and lipophilic gel useful for drug delivery or tissue coating. Additional background art includes U.S. Pat. No. 5,035,891, and US 2004/0247672. International Patent Application WO 2012131678, to some of the inventors, relates to sustained release formulations including poloxamers in a suspension form or other form of undissolved active agent, such that the disclosed formulations enable the use of higher amounts of the active agent within a single administration, while maintaining acceptable volumes of the administered dose.

Florfenicol is a commonly used broad-spectrum antibiotic agent, used for the treatment of Swine Respiratory Diseases (SRD) among other uses. The approved veterinary products of florfenicol include injectable formulations usually containing 300 mg/ml. One of such approved product for said injectable formulation for veterinary use is dissolved in an organic solvent N-methyl pyrrolidone (NMP). Some formulations for sustained release of florfenicol were previously disclosed, including Chinese Patent Application CN103202802, directed to sustained release formulations which include poloxamers and polysaccharides. The disclosure relates to several different polysaccharides and varied loadings of the active agent florfenicol in said formulations. A pharmacokinetic study of an in-situ forming gel for controlled delivery of florfenicol in pigs was disclosed in Geng et. al. [J. vet. Pharmacol. Therap. 38, 596-600], and demonstrated the increase in half-life of florfenicol in the animal plasma upon administration of 20% loading gels based on poloxamers and cellulose-based polysaccharide.

There is a need in the art to provide injectable formulations of antibiotics that could release the drugs in controlled manner over extended time intervals. There is a further need in the art to provide such formulations that would successfully maintain minimal inhibitory concentration levels for a variety of veterinary pathogens. There is a yet further need in the art to provide antibiotic formulations with high drug loading, e.g. above 25% to about 50%, which are yet still injectable via regular syringes.

### SUMMARY OF THE INVENTION

The stability of a sustained release formulation and the effect said stability has on the active agent release profile in the target organism over time is a crucial factor, which in many cases was proved to be a delicate balance between the different components in the formulation. It was surprisingly found, that utilizing a combination of a poloxamer, organic solvent and optionally a cellulose derivative which is at least partially soluble in organic solvents, in a sustained release formulation of an antimicrobial agent give rise to a stable injectable dispersion formulation, having a consistent and reproducible release profile, both in vitro and in vivo. Thus, is one aspect, the present invention provides a composition comprising a poorly soluble antimicrobial agent, at least one poloxamer, an organic solvent, and a cellulose derivative which is at least partially soluble in organic solvents, and an aqueous medium, wherein said composition is injectable. It was further surprisingly found that at very high loading of the active material e.g. above 35 wt % or 40 wt %, the combination of poloxamer and organic solvent in water may be sufficient to provide an injectable formulation having a consistent and reproducible release profile. Thus, is further aspect, the present invention provides a composition comprising an antimicrobial agent, at least one poloxamer, an organic solvent, and an aqueous medium, wherein the concentration of said antimicrobial is above 35 wt % to above 40 wt %, and wherein said composition is injectable.

Thus, provided herein a pharmaceutical composition comprising a biologically active agent, poloxamer, an aqueous carrier, and an organic co-solvent, wherein said composition is an injectable composition at room temperature, with a proviso that wherein said active agent concentration is below 35 wt % the composition further comprises a cellulose-based material which is at least partially soluble in organic solvents. In one embodiment, when the concentration of the drug is above 35 wt %, e.g. from 35 wt % and up to 50 or 55 wt %, the cellulose-based material is included. In further embodiments, when the concentration of the drug is above 35 wt %, e.g. from 35 wt % and up to 50 or 55 wt %, the composition is devoid of cellulose-based material, e.g. between 40 wt % and 50 wt %, or between 42.5 wt % and 50 wt %, or between 45 wt % and 50 wt %. Also provided herein a pharmaceutical composition comprising a biologically active agent, poloxamer, an aqueous carrier, an organic co-solvent, and a cellulose-based material which is at least partially soluble in organic solvents, wherein said composition is an injectable composition at room temperature, and wherein a concentration of said biologically active agent is above 10 wt %, and up to 35 wt %. The biologically active agent may be selected from florfenicol, lincomycin, tylosin, metronidazole, tilmicosin, spiramycin, erythromycin, tulathromycin, tiamulin, ampicillin, amoxicillin, clavulanic acid, penicillin, streptomycin, trimethoprim, sulfonamide, sulfamethoxazole, pleuromutilin, avilosin, tylvalosin, doxycycline, and oxytetracycline. Preferably, the biologically active agent is florfenicol. Further preferably, florfenicol may be present in the composition in a loading of between about 25 wt % to about 50 wt %. The organic co-solvent may be present at an amount of between about 5 to about 15% wt. The cellulose-based material which is at least partially soluble in organic solvents may be hydroxypropyl cellulose. The organic solvent may be selected from the group consisting of N-methyl pyrrolidone (NMP), dimethyl sulfoxide (DMSO), PEG 400, propylene glycol, and ethanol. Preferably, the organic solvent is N-methyl pyrrolidone. In some preferred embodiments, the pharmaceutical composition comprises the organic solvent which is N-methyl pyrrolidone, and the cellulose-based material which is at least partially soluble in organic solvents is hydroxypropyl cellulose, and biologically active agent is florfenicol at a concentration of at between 25 wt % and 50 wt %. In some other preferred embodiments, the pharmaceutical composition comprises the organic solvent which is N-methyl pyrrolidone, and florfenicol in a concentration between 35 wt % and 50 wt %. Also provided herein a pharmaceutical composition as defined herein for use in treating of a veterinary infection in a non-human animal by administering to said animal a pharmacologically effective dose of an antibiotic in said composition. Preferably, the composition is administered once to said non-human animal per the course of treatment. Further preferably, the administration comprises intramuscular injection, or subcutaneous injection. In some embodiments, the infection may be caused by a swine pathogen.

### BRIEF DESCRIPTION OF THE DRAWINGS

FIG. 1 schematically represents the release profiles of florfenicol from selected compositions.

FIG. 2 schematically represents the release profiles of florfenicol as the effect of added organic solvent.

FIG. 3 represents the blood plasma concentrations of florfenicol following single administration of a composition according to the invention, versus two administrations of a commercial product.

FIG. 4 represents the blood plasma concentrations of florfenicol following single administration of further compositions according to the invention, versus two administrations of a commercial product.

### DETAILED DESCRIPTION OF THE INVENTION

As described above, the sustained release composition of the present invention comprises an active biological agent. In some embodiments, said biological agent is preferably an antimicrobial agent, which demonstrates a poor solubility in aqueous media. The poor solubility may be understood as defined, e.g. in the current United States Pharmacopeia, but may be better understood in the context of the formulation, as explained in more detail below. In a related embodiment, the antimicrobial agent utilized in the sustained release composition of the invention is selected from the group consisting of florfenicol, lincomycin, tylosin, metronidazole, tilmicosin, spiramycin, erythromycin, tulathromycin, tiamulin, ampicillin, amoxicillin, clavulanic acid, penicillin, streptomycin, trimethoprim, sulfonamide, sulfamethoxazole, pleuromutilin, avilosin, tylvalosin, doxycycline, and oxytetracycline. In some currently preferred embodiments, the antimicrobial agent is florfenicol.

According the principles of the present invention, the loading (i.e. the amount of biologically active agent or antimicrobial agent which is introduced in the injectable dosage form) is high, allowing prolonged and controlled release over several days. The high loading of the injectable composition of the invention is promoted, among other factors, by having a formulation comprising a biologically active agent which may be in an insoluble form, thereby forming a dispersion in the aqueous medium. According to the principles of the invention, the antibacterial agent dispersed in the formulation is, to some extent, in a solid form. Preferably, more than 90% of the drug is in insoluble form, but the drug may be as much as 99.999% in an insoluble form. The insoluble form of the drug usually includes base compounds, or salts particularly having low water solubility, even if a more soluble salt may be known.

Depending on the solid-state properties of the active agent, the loading may vary. When the drug readily interacts with the aqueous medium or with poloxamer or other surface-active agents, it may form a paste, i.e. a composition that is not readily uptaken with a syringe (non-syringeable) and/or not injectable, at high loading values. In these cases such drugs may be used at rather low loading values, e.g. between 12 and 20% wt, but generally preferably the drug loading is high. Thus, in some embodiment the loading is at least about 20 wt % of the injectable composition.

In some other embodiments, the loading is between about 25 to about 30 wt % of the injectable composition. In some other embodiments, the loading is at least about 30 wt % of the injectable composition. In some further embodiments, the loading is between about 30 to about 45 wt % of the injectable composition. In some further embodiments, the loading is between about 35 to about 50 wt % of the injectable composition. In some embodiments, the loading is between about 30 to about 35 wt % of the injectable composition. In some specific embodiments, when the biologically active agent is florfenicol, florfenicol loading used for a specific applications may be between 25 and 50 wt %, such as between 28 and 32 wt %, or between 36 and 42 wt %, or between 44 and 48 wt %.

The biologically active agent forms dispersion in the aqueous medium with the co-solvent. It is understood that the biologically active agent should be in a form of solid, e.g. powder. The powder may be in form of aggregates, granulation, or coated powder, but preferably the powder is neat drug substance powder, of a defined particle size distribution. In some preferred embodiments, the powder has the particle size of less than about 90 microns, more preferably less than about 50 microns. It may sometimes be advantageous also to use smaller particle sizes, or even micronized powder. Without being bound by a theory it is believed that powder of smaller particle size may increase the peak plasma concentration obtainable from a formulation in vivo, in comparison to regular drug powder, even if in vitro the difference would be small or insignificant. Micronized powder or powder with reduced particle size may be obtained directly from the powder of the biologically active substance, as generally known in the art, e.g. by high-impact or high-shear milling, sieving under pressure, and in other ways.

In certain preferred embodiments, the biologically active material or antimicrobial agent is released from the in-situ formed gel of the composition of the present invention during at least 3 days. In some other embodiments, the material is being released over between 2 to 3 days. In some further embodiments, the material is being released over between 4 to 5 days. In some embodiments, the material is being released over more than 5 consecutive days from a single injectable composition of the invention. The release may be thus described in terms of release duration rather than any specific rate. The duration of the release in vivo may be detected in the plasma as the drug concentrations maintaining significant levels over time. In another embodiment, the duration of the release in vivo may be detected in the target organ or tissue as the drug concentrations maintaining significant levels over time. In particular, insofar the active agent is an antibiotic, the duration of the release may be detected in blood plasma, and the concentrations obtained may be compared to the minimum inhibitory concentrations of the antibiotics for specific pathogens. In vitro, due to the maintenance of sink conditions, the duration of the drug release may be from about 12 hours to about 3 days, e.g. in the conditions as described in the Examples section below.

According to some of the principles of the present invention, the advantageous combination of organic co-solvent, poloxamer in aqueous medium, and a cellulose derivative which is at least partially soluble in organic solvents, give rise to a synergistic effect, allowing a stable and controlled release of the biologically active agent, over several days. The drug loading in the formulations comprising such cellulose derivative may be as low as about 5 wt %, or about 10 wt %. However, depending on the antibiotic solid-state properties, the drug loading may be as high as 35 wt %, or 40 wt %, or 45 wt %, or 47.5 wt %, or even 50 wt %. Moreover, when the active agent is present in a concentration of above 35 wt %, it has been unexpectedly found that relatively stable and repeatable drug release kinetics may be achieved from compositions comprising poloxamer, water and an organic co-solvent as defined herein. Whereas the presence of the cellulose derivative which is at least partially soluble in organic solvents was found beneficial even at high drug loading, the release profiles without the excipient were surprisingly consistent enough to meet the requirements of the current United States Pharmacopiea for the variability of the drug release of the controlled-release dosage forms. When the drug loading is below 35 wt %, however, it is preferable that the composition comprise the cellulose derivative as describes below.

According to some embodiments, the poloxamer as described above is selected from the group consisting of poloxamer 407, poloxamer 188, poloxamer 237 and poloxamer 338, and combination thereof. In some currently preferred embodiments, the poloxamer as described above is poloxamer 407.

The presence of poloxamer allows the composition to gel under physiological temperature, and hence, said poloxamer must exist in a suitable concentration in the injectable composition to enable the formation of a stable gel, particularly in presence of a large amount of the undissolved powder of the active agent. Accordingly, the concentration of the poloxamer as described above is above 8 weight percent from the total weight of the formulation. Depending on the nature of the drug, e.g. the particle size, drug solubility, its affinity towards poloxamer, and on the loading of the drug, the amount of poloxamer may be as low as 7 to 9 wt % and up to 16 to 20 wt %.

The synergistic effect of some of the embodiments of the present invention is achieved by combining said poloxamer with a unique combination of an organic co-solvent and a cellulose derivative which is at least partially soluble in organic solvents. The chemical compatibility between the cellulose derivative and the organic solvent, and the ratio between these two components determine, together with the poloxamer concentration, the release profile of the biologically active agent. Without being bound by any mechanism or theory, it is postulated that while the organic solvent may be increasing the solubility of the biologically active agent, it also slows down the release rate of said active agent from the gel-form composition under physiological conditions, due to its effect on the gel itself. It is further postulated that at least for some drugs, the addition of the cellulose derivative as described above may be responsible for the increase in the biologically active agent release rate, and that the organic solvent contributes to a reduced variability in the overall release profile over time. Although the molecular weight of the cellulose derivative to be used may be selected according to the rheological properties required and the contemplated release profile, according to the some embodiments of the present invention, the concentration ratio between said cellulose derivative and said organic solvent may usually be between about 1:6 to about 1:20. When the drug is present in particularly high loading, e.g. above 35 wt % to above 40% wt, the concentration ratio between said cellulose derivative and said organic solvent may be between about 1:10 to about 1:100.

The cellulose derivative which is at least partially soluble in organic solvents is usually such that it dissolves to some appreciable extent in common pharmaceutical organic solvents, e.g. in ethanol. Preferably, the suitable derivative forms a clear solution upon dissolution of, e.g. 1 gram of the derivative in 100 mL of 96%-ethanol at room temperature. One suitable cellulose derivative which is at least partially soluble in organic solvents is hydroxypropyl cellulose. Hydroxypropyl cellulose possesses a further useful property that it is also highly soluble in aqueous solutions at room temperatures and becomes less soluble with increased temperature. Without being bound by any theory or mechanism of action, it is postulated that upon injection of the composition of the invention into the animal, the solubility of hydroxypropyl cellulose decreases, which in turn contributes to the stability of the formed gel, resulting in a better control over the release of the biologically active agent.

In some related embodiments, the concentration of the cellulose derivative as described above is between about to about 0.5 wt % to about 1.5 wt % of the total weight of the injectable composition. In some other embodiments, the cellulose derivative concentration is between about 0.5 to about 1 wt %. When the drug is present in a very high loading, e.g. above 40%, the cellulose derivative concentration may be between about 0.05 to about 0.7 wt %.

In some embodiments, the organic solvent as described above is selected from the group consisting of N-methyl pyrrolidone (NMP), dimethyl sulfoxide (DMSO), PEG 400, propylene glycol, and ethanol. In some currently preferred embodiments, the organic solvent is NMP.

In some related embodiments, the concentration of the organic solvent as described above is between about to about 1.5 wt % to about 20 wt % of the total weight of the injectable composition. In some other embodiments, the organic solvent concentration is between about 3 to about 15 wt %. In yet some other embodiments, the organic solvent concentration is between about 8 to about 12 wt %.

In further related embodiments, at least one poloxamer, an organic solvent, and the cellulose derivative, are dissolved in an aqueous medium. The aqueous medium is usually water, optionally comprising further dissolved additives, such as salts and/or buffers. The amount of the aqueous medium in the preparation is usually the remainder from the 100% of the composition upon subtraction the respective percentages of the biologically active agent, the at least one poloxamer, the cellulose derivative, the co-solvent, and other excipients if used. The salts may include sodium chloride, calcium chloride, or magnesium chloride, and the buffers may include mono-, di-, or tri-basic salts of alkali metals and phosphates.

Whereas the synergistic effect that may be present for the co-solvent, the cellulose derivative which is at least partially soluble in organic solvents, and poloxamer in an aqueous medium is clearly beneficial, when the drug is present in very high loading, e.g. above 35 wt % to above 40 wt %, the effect of cellulose derivative on the stabilization of the system may become less required to obtain a pharmaceutically acceptable composition, e.g. demonstrating the release profile with the relative standard deviation in the concentrations' values at each time point of below 10%. As demonstrated in the examples below, e.g. the omission of hydroxypropyl cellulose from a formulation of florfenicol at a loading of 47.5 wt % resulted in a mild burst effect with the increase of the relative standard deviation (RSD) at early time points, but also in an acceptable release profile.

According to the principles of the invention, the formulation achieved is a stable and injectable formulation at room temperature (e.g. between 15° C. and 25° C.), or on cold (e.g. between 2° C. and 8° C.), which upon injection into the animal body (e.g. having a temperature above 35° C.) transforms into a gel form, characterized in having a reproducible and well-controlled release profile of the biologically active agent incorporated therein.

In another aspect, the present invention provides a preparation method of injectable sustained release formulations comprising antimicrobial agent, at least one poloxamer, an organic solvent, and a cellulose derivative which is at least partially soluble in organic solvents, in an aqueous medium, having the steps of: 1) mixing water and organic solvent (known as co-solvent) and preferably cooling the resultant mixture, 2) adding consecutively or concomitantly the at least one poloxamer and said cellulose derivative into the [cold] mixture of step 1, followed by mixing until dissolution; and 3) adding the antimicrobial agent into the resultant mixture.

In some embodiments, the organic solvent as described above is selected from the group consisting of N-methyl pyrrolidone (NMP), DMSO, PEG 400, propylene glycol, and ethanol. In some currently preferred embodiments, the organic solvent is NMP.

In some embodiments, the poloxamer as described above is selected from the group consisting of poloxamer 407, poloxamer 188, poloxamer 237, poloxamer 338, and combination thereof. In some currently preferred embodiments, the poloxamer as described above is poloxamer 407.

In some embodiments, the cellulose derivative is hydroxypropyl cellulose.

In some embodiments, the antimicrobial agent utilized in step 3 is selected from the group consisting of florfenicol, lincomycin, tylosin, metronidazole, tilmicosin, spiramycin, erythromycin, tulathromycin, tiamulin, ampicillin, amoxicillin, clavulanic acid, penicillin, streptomycin, trimethoprim, sulfonamide, sulamethoxazole, pleuromutilin, avilosin, tylvalosin, doxycycline, oxytetracycline. In some currently preferred embodiments, the antimicrobial agent is florfenicol.

The term “biologically active agent” as appears herein and in the claims is interchangeable with the term “antibacterial agent”, “drug” or “antibiotics”.

As appears herein and in the claims the term “co-solvent” refers to the organic solvent which is mixed with the aqueous carrier or water in the formulation of the invention. In some embodiments, the organic solvent as described above is selected from the group consisting of N-methyl pyrrolidone (NMP), DMSO, PEG 400, propylene glycol, and ethanol.

In a further aspect there is provided a method of treatment of veterinary infections, or use of the compositions in treating of the veterinary infections, by administering to a patient in need thereof at least one injection of an injectable sustained release compositions as generally described herein, comprising, in an aqueous medium, an antimicrobial agent, at least one poloxamer, an organic solvent, and optionally a cellulose derivative which is at least partially soluble in organic solvents. Preferably, the method comprises a single administration of the formulation, but more than one injection may be used according to the need and the length of the treatment. In dealing with a veterinary patient it is advantageous to minimize the handling, so that to decrease the animal distress and the effort required to locate, trap and handle the sick animal. Therefore, an administration on a single occasion is preferred. Alternatively, the method comprises multiple administrations of the formulation, as long as the number of administrations is lower than currently required for the specific biologically active agent.

The administration may include a single injection, or multiple injections into multiple sites, if a large volume of the injection is required. Due to the advantages of the formulations of the present invention, it may not be necessary to use multiple injection sites, as the poorly-soluble drug is present in sufficient amount in relatively small volumes of the injection.

The administration is usually an intramuscular injection. However, the administration may also be a subcutaneous administration, intraperitoneal administration, intradermal administration, or specific administration sites, such as intravulval administration for cows and sheep, intracaudal or ear administration for beef cattle, intramammary, and the like.

The veterinary infections that may be treated according to the invention include the infections caused by the pathogens of swine, infections of cattle, infections of poultry, infections of companion animals, or infections of zoo- and wildlife animals.

In some embodiments, the organic solvent as described above is selected from the group consisting of N-methyl pyrrolidone (NMP), DMSO, PEG 400, propylene glycol, and ethanol. In some currently preferred embodiments, the organic solvent is NMP. In some embodiments, the poloxamer as described above is selected from the group consisting of poloxamer 407, poloxamer 188, poloxamer 237, poloxamer 338, and combination thereof. In some currently preferred embodiments, the poloxamer as described above is poloxamer 407. In some embodiments, the cellulose derivative is hydroxypropyl cellulose. In some embodiments, the antimicrobial agent is selected from the group consisting of florfenicol, lincomycin, tylosin, metronidazole, tilmicosin, spiramycin, erythromycin, tulathromycin, tiamulin, ampicillin, amoxicillin, clavulanic acid, penicillin, streptomycin, trimethoprim, sulfonamide, sulfamethoxazole, pleuromutilin, avilosin, tylvalosin, doxycycline, oxytetracycline. In some currently preferred embodiments, the antimicrobial agent is florfenicol.

### EXAMPLES

### Materials and Methods

Florfenicol and N-methylpyrrolidone (NMP) were purchased from Sigma-Aldrich, Israel. Poloxamers, 407, 188, 338 and 237 were obtained from local representative of BASF. Amoxicillin, tylosin, Klucel® polymers (hydroxypropyl cellulose), PEG400, and propylene glycol were obtained as a gift from pharma companies. Water was purified on a column and distilled before use. Sodium chloride was purchased from Merck, Israel.

Unless indicated otherwise, florfenicol injectable formulations were prepared as follows:

Weighed quantities of water and co-solvent were mixed at room temperature, and salts or buffers, if present in the formulation, were added and mixed to achieve dissolution. Weighed amounts of poloxamer and cellulose derivative were cooled to 4° C. in a cold room; separately, water and co-solvent mixtures were cooled too. The polymers were then added to the water and co-solvent mixture under the same conditions, and were vigorously mixed using a magnetic stirrer, until a clear solution was obtained. Florfenicol powder (flakes) was then added to the resulted solution and mixed for 24 hours in a cold room, to ensure good distribution in the preparation. Alternatively, particularly for high-loading formulations, a weighed amount of florfenicol was placed in a mortar, and geometrically levigated, i.e. mixed in a mortar with comparable aliquots of the solution, until all of the weighed aliquot of prepared solution was used up.

### Measurement of the Gelation Point

The gelation was measured by inverting a glass tube containing 0.5-1 mL of the formulation, at increasing temperatures. The temperature whereat the formulation stopped flowing down upon inversion was considered a primary gelation point. Alternatively, for preliminary screening, the temperature was elevated to 40° C. and the time it took the formulation to become a gel-form was recorded.

The gelation point was also measured rheometrically, using Anton Paar Rheometer Physica MCR 101, parallel plate spindles separated with 200 μm gap, with a temperature sweep at shear rate of 100 reciprocal seconds. Second derivative of the viscosity curve furnished the sharpest change of in viscosity, which was considered as the true gelation point.

### Determination of Florfenicol

Florfenicol was determined using HPLC, using HP1090 apparatus, with UV detector measuring absorbance at 224 nm. A C-18 250×4.6 5 μm column was used, with elution at 1.2 ml/min, with 25:75 ACN:DDW mobile phase. Florfenicol eluted under these conditions at 4.-4.5 minutes.

### Dissolution Testing

To test the dissolution kinetics of florfenicol from the formulations, a syringe barrels of 5-mL syringes, were cut into 2-mL segments to serve as holders—in a shape of a tube. One side was closed with Parafilm® sheet, and about 2-mL aliquots of the formulation at room temperature were accurately weighed into said prepared tube-holders, through 19 G needle using a suitable syringe, thus evaluating the injectability of the formulation. The top side was then closed with another Parafilm® sheet and placed into a pre-heated oven to 40° C., for at least 15 minutes to ensure gelation. The Parafilm® sheets were then accurately removed, the tube-holder was placed into a sinker basket and immediately transferred into Caleva 6ST dissolution tester (USP Apparatus 2), set to 20 rpm at 40° C. The temperature was chosen to fit and mimic the body temperature of the target animal (swine). The dissolution medium was phosphate buffer USP, at pH 6.8, and a 900 mL volume was used per tube-holder. Samples were drawn from the dissolution medium at predetermined times, and the volume was corrected with fresh dissolution medium. At the end of the test, the tube-holders were washed in the dissolution vessels and vigorously mixed to obtain the recovery amount of the material to serve as the 100% reference. The percentile of maximal concentration of florfenicol at each time point with the standard deviation was reported.

Additionally, the dissolution of some of florfenicol compositions was performed using USP apparatus 5 (paddle over disc), as indicated below.

### Example 1—Comparative Example

A. In order to evaluate the efficiency of the disclosed formulations in Chinese patent application CN103202802, Example 7 (30% florfenicol) of said publication was reproduced and tested under the described conditions. As the publication contains little guidance as to the grade of hypromellose used, two grades having an apparent viscosity of below 20 cP at the tested low concentrations (HPMC K4M and HPMC K15M) were tested separately. Briefly, the poloxamers were accurately weighed, cooled and dissolved in a large portion of cold water at 4° C., followed by the addition of hydroxypropyl methyl cellulose (HPMC). The rest of the excipients were provided from stock solutions, and the remainder water content was added and thoroughly mixed. Formulation samples having total quantities of 25 grams were prepared, samples prepared utilizing HPMC K4M are referred to as sample preparation 1.1 and samples prepared utilizing HPMC K15M are referred to as sample preparation 1.2.

To test the advantageous effect of the organic solvent according to the present invention, the same formulations as described above were prepared, this time using ca. 20 wt % of N-methyl pyrrolidone as a co-solvent, of the total solvent weight (replacing 20% of the water with an organic solvent), thereby obtaining sample preparation 1.3 and sample preparation 1.4, corresponding to HPMC K4M and HPMC K15M, respectively.

It was found out that under the experiment conditions, i.e. at room temperature, samples prepared according to 1.1 and samples prepared according to 1.2 could not be pulled into a syringe even without a needle. This is to show said formulation prepared according to the disclosure of CN103202802 (Example 7) appeared to be not-injectable under the reported conditions. In order to gain the release profile and results of said non-injectable formulations, the samples were supplied utilizing a spatula. It should be further mentioned that the addition of NMP as a co-solvent increased the viscosity beyond practical (hard gel even at 4° C.), however, sample prepared according to 1.3 and 1.4 were tested for the drug release, despite that they could not be injected as well.

B. In order to produce injectable compositions, 20% loading formulations were produced, following the trend of the Example 7 and Example 6 of the prior art publication CN103202802. Briefly, the florfenicol loading was decreased on account of water. Sample preparation 1.5 included HPMC K15M and pure water and sample preparation 1.6 included HPMC K15M and 20 wt % NMP as a co-solvent. The resultant formulations according preparations 1.5 and 1.6 having 20 wt % florfenicol were easily injected via the tested needle and gelled under sample preparation conditions for the dissolution testing.

To test the release profile of florfenicol from the formulations described above despite the lack of injectable properties of compositions prepared according to 1.1-1.4, said compositions were applied to the tubes using a spatula in a usual circular semisolids' filling technique. The results are presented in Table 1 below.

It can be seen from Table 1 that generally the addition of NMP to the composition comprising HPMC accelerates the release rate of florfenicol from the preparations, and sometimes decreases the variability, e.g. when comparing the preparations 1.1 with 1.3, 1.2 with 1.4, and 1.5 with 1.6.

It can equally be seen that the injectable formulation according to the prior art publication can have only 20% loading of florfenicol, which is also evidenced by another publication of the same inventors, Z. X. Geng, H. M. Li, J. Tian, T. F. Liu, Z. G. Yu, J Vet Pharmacol Ther, Vol 38, Iss 6, December 2015, 596-600). Higher loading could not be accomplished as injectable compositions utilizing the formulation according to the prior art.

TABLE 1

| | Preparation | | Preparation | | Preparation | | Preparation | | Preparation | | Preparation | |
|---|---|---|---|---|---|---|---|---|---|---|---|---|
| Time | 1.1 | | 1.2 | | 1.3 | | 1.4 | | 1.5 | | 1.6 | |
| (hr) | mean | sd | mean | sd | mean | sd | mean | sd | mean | sd | mean | sd |
| 0.25 | 2.52 | 0.51 | 2.61 | 0.44 | 2.61 | 0.44 | 2.98 | 0.38 | 2.47 | 2.15 | 3.94 | 0.61 |
| 0.5 | 4.25 | 1.16 | 5.80 | 0.79 | 5.80 | 0.79 | 7.39 | 0.47 | 5.52 | 4.35 | 9.34 | 0.49 |
| 1 | 6.54 | 1.49 | 11.02 | 2.12 | 11.02 | 2.12 | 15.50 | 1.06 | 10.41 | 7.06 | 15.25 | 1.15 |
| 1.5 | 8.34 | 1.45 | 15.44 | 2.92 | 15.44 | 2.92 | 22.30 | 1.78 | 15.76 | 7.09 | 20.28 | 1.48 |
| 2 | 9.43 | 1.60 | 18.70 | 3.28 | 18.70 | 3.28 | 27.90 | 2.65 | 19.59 | 6.84 | 24.13 | 1.81 |
| 3 | 12.56 | 3.02 | 23.94 | 3.23 | 23.94 | 3.23 | 36.28 | 3.94 | 27.20 | 6.40 | 30.80 | 2.96 |
| 4 | 16.40 | 3.22 | 28.94 | 2.33 | 28.94 | 2.33 | 43.82 | 3.11 | 32.61 | 5.57 | 37.23 | 4.07 |
| 5 | 20.63 | 5.31 | 35.19 | 4.10 | 35.19 | 4.10 | 49.79 | 2.01 | 36.75 | 4.77 | 42.60 | 4.02 |
| 6 | 23.73 | 5.72 | 38.59 | 1.59 | 38.59 | 1.59 | 55.42 | 2.43 | 41.78 | 6.86 | 48.17 | 3.10 |
| 7 | 27.12 | 6.67 | 42.20 | 1.93 | 42.20 | 1.93 | 57.69 | 1.87 | 43.69 | 4.76 | 53.70 | 2.96 |
| 8 | 30.22 | 7.38 | 45.46 | 1.71 | 45.46 | 1.71 | 63.62 | 5.77 | 46.27 | 4.46 | 59.21 | 5.93 |
| 24 | 48.68 | 9.42 | 72.25 | 7.96 | 72.25 | 7.96 | 87.11 | 7.07 | 75.23 | 8.86 | 85.66 | 7.62 |

| Max viscosity | 44.9 Pa*s | 38.4 Pa*s | | 18.3 Pa*s | 22.9 Pa*s |
|---|---|---|---|---|---|
| Min viscosity | 0.59 Pa*s | 0.49 Pa*s | Hard gel at 4° C. | 0.18 Pa*s | 0.27 Pa*s |
| Gelation t° C. | 12.9° C. | 12.8° C. | | 18.4° C. | 17.1° C. |
| Gelation range | 12.3-14.4 | 12.3-14.8 | | 17.5-19.9 | 16.2-19.9 |

### Example 2

In order to evaluate the advantages of the florfenicol sustained release formulation according to the principles of the present invention compared with another know gel-based sustained release formulation disclosed in International patent application WO2012131678, gels comprising 30% of florfenicol by weight were produced. The effect of the co-solvent NMP, the cellulose based material hydroxypropyl cellulose, and their synergistic combination were isolated and studied. All formulations demonstrated gelation between 25° C. and 35° C. (individual data given below), and the release profiles were evaluated according to the method above. The formulations are summarized in the tables below, together with their respective release profiles data.

Preparation 2.1 is according to an embodiment of the present invention and comprises both the cellulose based material hydroxypropyl cellulose; preparation 2.2 shows the effect of omission of the co-solvent; preparation 2.3 shows the effect of omission of hydroxypropyl cellulose (Klucel® EF) and the co-solvent NMP; and preparation 2.4 is a comparative preparation according to WO2012131678, having no co-solvent and no cellulose additive. Preparations 2.5 (of an embodiment of the invention) demonstrates a lower loading (20 wt % florfenicol) and 2.6 contain 20% of florfenicol and no NMP for comparison with preparations 2.6.

Upon comparing the results of preparation 2.1 and 2.3, it can be readily observed that the addition of NMP to the formulation according to WO 2012131678 causes a significant decrease in drug release, with a significant reduction of variability between the results. Additionally, the addition of hydroxypropyl cellulose to the formulation according to WO 2012131678 leads to significant reduction of the release rate and to relatively high variability in the release profile. According to the results, only the addition of both components (NMP and HPC) is responsible for the synergistic effect leading to lower variability (the standard deviation of the mean in relative to the mean is lower), and increases the drug release compared to purely aqueous preparation 2.2, and 2.3. Furthermore, it can be seen that the formulations having 20% loading according to the invention produce comparable yet somewhat more attenuated release of florfenicol with yet lower variability than the hypothetical 20% formulation of CN′802 with hypromellose instead of hydroxypropyl cellulose (preparation 1.5).

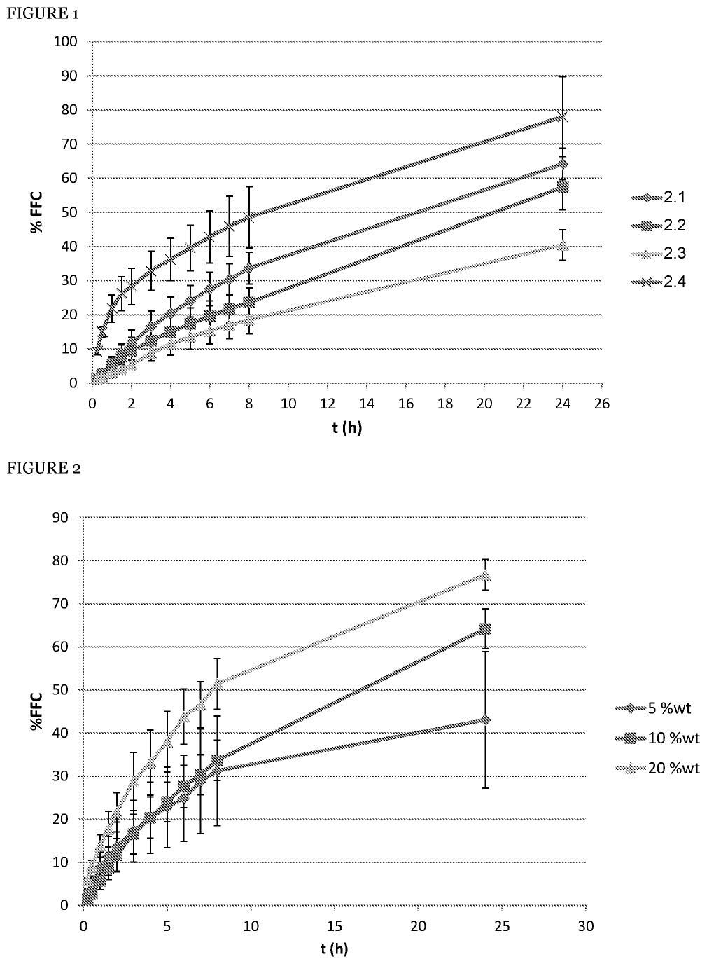

The results are summarized in the Table 2 below, and also in the FIG. 1. In the FIG. 1, the release profiles are demonstrated with the error bars indicating the RSD at every time point. The diamonds (♦) represent preparation 2.1, solid squares (▪) preparation 2.2, solid triangles (▴) preparation 2.3, and X-signs (x) preparation 2.4, with “% FFC” indicating the cumulative release percentile of florfenicol, and “t (h)” indicated time elapsed from the beginning of the experiment, in hours.

TABLE 2

| | Preparation | | Preparation | | Preparation | | Preparation | | Preparation | | Preparation | |
|---|---|---|---|---|---|---|---|---|---|---|---|---|
| | 2.1 | | 2.2 | | 2.3 | | 2.4 | | 2.5 | | 2.6 | |
| | Wt (g) | % w/w | Wt (g) | % w/w | Wt (g) | % w/w | Wt (g) | % w/w | Wt (g) | % w/w | Wt (g) | % w/w |
| Flor- | 7.50 | 30.00 | 7.50 | 30.00 | 7.50 | 30.00 | 7.50 | 30.00 | 5.00 | 20.00 | 5.00 | 20.00 |
| fenicol | | | | | | | | | | | | |
| Polo- | 3.00 | 12.00 | 3.00 | 12.00 | 3.13 | 12.50 | 3.13 | 12.00 | 3.43 | 13.72 | 3.43 | 13.72 |
| xamer | | | | | | | | | | | | |
| 407 | | | | | | | | | | | | |
| Klucel ® | 0.19 | 0.75 | 0.19 | 0.75 | — | — | — | — | 0.22 | 0.88 | 0.22 | 0.88 |
| EF | | | | | | | | | | | | |
| DDW | 11.81 | 47.25 | 14.31 | 57.25 | 11.81 | 47.50 | 14.38 | 57.50 | 13.50 | 54.00 | 16.36 | 65.44 |
| NMP | 2.50 | 10.00 | — | — | 2.50 | 10.00 | — | — | 2.86 | 11.44 | — | — |
| Time(hr) | mean | sd | mean | sd | mean | sd | mean | sd | mean | Sd | mean | sd |
| 0.25 | 1.37 | 0.50 | 1.33 | 0.56 | 1.05 | 0.52 | 9.29 | 1.05 | 1.75 | 0.87 | 4.40 | 1.55 |
| 0.5 | 2.80 | 0.98 | 2.75 | 1.24 | 1.72 | 0.65 | 14.97 | 1.44 | 3.46 | 0.31 | 5.66 | 2.81 |
| 1 | 5.70 | 2.02 | 5.21 | 2.30 | 2.89 | 1.11 | 21.85 | 4.01 | 7.34 | 0.62 | 9.69 | 4.11 |
| 1.5 | 8.76 | 2.76 | 7.64 | 3.57 | 3.96 | 1.43 | 26.20 | 4.98 | 11.06 | 0.74 | 12.18 | 4.95 |
| 2 | 11.67 | 3.86 | 9.42 | 4.03 | 5.48 | 1.23 | 28.28 | 5.34 | 15.90 | 1.11 | 13.85 | 5.75 |
| 3 | 16.53 | 4.56 | 12.39 | 4.45 | 8.71 | 2.23 | 32.91 | 5.76 | 20.70 | 1.08 | 16.29 | 7.76 |
| 4 | 20.39 | 4.78 | 15.01 | 4.77 | 11.42 | 3.22 | 36.29 | 6.21 | 26.25 | 1.17 | 18.84 | 8.52 |
| 5 | 24.00 | 4.61 | 17.34 | 4.72 | 13.53 | 3.75 | 39.61 | 6.67 | 30.54 | 1.56 | 21.09 | 9.93 |
| 6 | 27.59 | 4.94 | 19.64 | 4.45 | 15.30 | 3.88 | 42.83 | 7.66 | 33.59 | 1.10 | 23.24 | 8.98 |
| 7 | 30.34 | 4.63 | 21.80 | 4.20 | 16.93 | 3.97 | 45.93 | 8.81 | 36.57 | 0.64 | 27.32 | 8.40 |
| 8 | 33.67 | 4.68 | 23.61 | 4.24 | 18.49 | 4.00 | 48.57 | 9.02 | 39.74 | 0.71 | 30.22 | 8.09 |
| 24 | 64.18 | 4.63 | 57.38 | 6.57 | 40.47 | 4.47 | 78.06 | 11.68 | 66.69 | 5.16 | 59.45 | 12.58 |

Rheology

| | | | | | | |
|---|---|---|---|---|---|---|
| Max | 9.69 Pa*s | 12.3 Pa*s | 11.4 Pa*s | 13 Pa*s | 8.11 Pa*s | 8.3 Pa*s |
| viscosity | | | | | | |
| Min | 0.38 Pa*s | 0.262 Pa*s | 0.181 Pa*s | 0.346 Pa*s | 0.125 Pa*s | 0.0819 Pa*s |
| viscosity | | | | | | |
| Gelation | 27.1° C. | 24° C. | 27.6° C. | 23.9° C. | 27.9° C. | 25.3° C. |
| t° C. | | | | | | |
| Gelation | 27.0-31.6 | 23.6-27.1 | 27-31.2 | 23.5-27.1 | 27.5-31.2 | 24.4-27.5 |
| range | | | | | | |

### Example 3

In order to evaluate the effect of the co-solvent of choice, NMP, on the formulation, gels according to the preparation 2.1 were produces, and NMP content was varied from 5 to 20 weight percent, to furnish preparation 3.1 (5% wt) and 3.2 (20% wt).

The release data are presented in the table 3 below, and the profiles are demonstrated in the FIG. 2, with the error bars indicating the RSD at every time point. The diamonds (♦) represent preparation 3.1 (designated as “5 wt %”), solid squares (▪) preparation 2.1 (designated as “10 wt %”), and solid triangles (▴) preparation 3.2 (designated as “20 wt %”), with “% FFC” indicating the cumulative release percentile of florfenicol, and “t (h)” indicated time elapsed from the beginning of the experiment, in hours.

It can be seen that at 5% wt of NMP the variability increases while the release profile remains almost unchanged, whereas with 20% the release is slightly accelerated.

TABLE 3

| | Preparation 3.1 | | Preparation 3.2 | |
|---|---|---|---|---|
| Time (hr) | mean | sd | mean | sd |
| 0.25 | 2.79 | 1.06 | 5.92 | 0.48 |
| 0.5 | 5.05 | 1.73 | 9.14 | 1.31 |
| 1 | 8.88 | 3.45 | 13.81 | 2.57 |
| 1.5 | 11.49 | 4.53 | 17.69 | 4.17 |
| 2 | 13.67 | 5.70 | 21.63 | 4.57 |
| 3 | 17.21 | 7.16 | 28.77 | 6.72 |
| 4 | 20.37 | 8.25 | 33.16 | 7.55 |
| 5 | 22.74 | 9.32 | 37.94 | 7.03 |
| 6 | 24.86 | 10.01 | 43.80 | 6.41 |
| 7 | 28.79 | 12.15 | 46.57 | 5.34 |
| 8 | 31.25 | 12.73 | 51.41 | 5.88 |
| 24 | 43.06 | 15.81 | 76.69 | 3.56 |

Rheology

| | | |
|---|---|---|
| Ma viscosity | 6.16 Pa * s | 10.9 Pa * s |
| Min viscosity | 0.23 Pa * s | 0.39 Pa * s |
| Gelation t° C. | 26.4° C. | 21.6° C. |
| Gelation range | 26.2-29.3 | 21.1-24.7 |

### Example 4

In order to evaluate the effect of additional co-solvents on the formulation, gels according to the preparation 2.1 were produced, and NMP was substituted with either DMSO (preparation 4.1), propylene glycol (preparation 4.2), PEG 400 (preparation 4.3), or ethanol (preparation 4.4).

The release profiles are summarized in the Table 4 below.

It can be readily seen that both DMSO and PEG 400 give comparable release profile with NMP, but decrease significantly more the gelation point of the solution.

TABLE 4

| | Preparation 4.1 | | Preparation 4.2 | | Preparation 4.3 | | Preparation 4.4 | |
|---|---|---|---|---|---|---|---|---|
| Time(hr) | mean | sd | mean | sd | mean | sd | mean | sd |
| 0.25 | 2.27 | 0.36 | 1.38 | 0.37 | 2.44 | 0.69 | 1.53 | 0.80 |
| 0.5 | 4.44 | 0.70 | 2.36 | 0.90 | 4.86 | 1.35 | 2.57 | 1.39 |
| 1 | 8.61 | 1.18 | 3.65 | 1.36 | 9.52 | 2.57 | 3.94 | 1.96 |
| 1.5 | 11.14 | 1.00 | 4.44 | 1.59 | 12.93 | 3.94 | 4.92 | 2.27 |
| 2 | 13.09 | 0.92 | 5.34 | 1.63 | 15.28 | 4.50 | 5.86 | 2.45 |
| 3 | 15.98 | 1.59 | 6.89 | 1.72 | 19.62 | 6.25 | 7.66 | 2.82 |
| 4 | 18.70 | 2.62 | 8.14 | 1.91 | 23.53 | 7.55 | 9.17 | 3.37 |
| 5 | 21.61 | 4.36 | 9.61 | 2.08 | 27.58 | 8.78 | 10.84 | 3.61 |
| 6 | 24.26 | 5.55 | 10.82 | 2.05 | 30.58 | 9.32 | 12.20 | 3.72 |
| 7 | 27.28 | 6.75 | 12.45 | 2.21 | 33.89 | 9.78 | 13.97 | 4.29 |
| 8 | 30.14 | 7.61 | 13.70 | 2.42 | 36.84 | 10.12 | 15.49 | 5.06 |
| 24 | 68.9 | 7.22 | 32.8 | 4.48 | 69.36 | 4.96 | 36.31 | 11.66 |

Rheology

| | | | | |
|---|---|---|---|---|
| Max viscosity | 10.5 Pa*s | 10.5 Pa*s | 8.16 Pa*s | 4.25 Pa*s |
| Min viscosity | 0.29 Pa*s | 0.37 Pa*s | 0.35 Pa*s | 0.21 Pa*s |
| Gelation t° C. | 15.1° C. | 22.0° C. | 20.2° C. | 33.8° C. |
| Gelation range | 14.7-17.0 | 21.6-24.7 | 19.4-22.9 | Broad |

### Example 5

To demonstrate the effect of the invention in vivo, a pharmacokinetic study was performed to demonstrate prolonged and effective plasma levels from a single administration of florfenicol in pigs. The study has been approved by the Ethics Committee for Animal Research studies in the Hebrew University of Jerusalem. A total of six animals were used with two female pigs of 3-4 months of age. A 20 G central vein catheter was inserted into a jugular vein of each pig to facilitate blood collection. All animals received 40 mg/kg of the one-shot treatment of the preparation 2.1 in the first arm of the study, and either 20 mg/kg as Nuflor® (Merck Animal Health—florfenicol 30% solution in NMP) given twice 48 h apart, or a different test treatment in the second arm, after a wash out period of two weeks.

Blood samples were withdrawn before each treatment administration (time 0) and at 1, 2, 4, 6, 8, 10, 24, 30, 52, 72, 96, 144 and 196 hours after first administration. The samples were collected into heparinized tubes and plasma was immediately separated and stored at −20° C. till analysis. On the day of the analysis the samples were spiked with internal standard (chloramphenicol) and extracted with acetonitrile. Standards were prepared on the same day. Determination of parent drug, florfenicol, and the main metabolite, florfenicol-amine, was done using UHPLC-MS/MS (TSQ Quantum Access Max mass spectrometer in positive ion mode using electron spray ionization (ESI) and multiple reaction monitoring (MRM) mode of acquisition in duplicate. Results for florfenicol (parent compound) and for florfenicol-amine (main metabolite) were obtained.

The analysis of the data was performed using Microsoft Excel software. The area under the curve values (AUC) were obtained by trapezoidal rule. The terminal slopes were identified by semilogarithmic transformation, and the slope was calculated by fitting the curves to exponential decline data. All further calculations were performed with the fitted functions. No deconvolution was performed due to complexity of the model, particularly for double-injection arms. For these Nuflor® arms, the terminal slope data was also used to extrapolate the 48-hours points. The data were calculated from an average curve; range of individual values is presented where applicable.

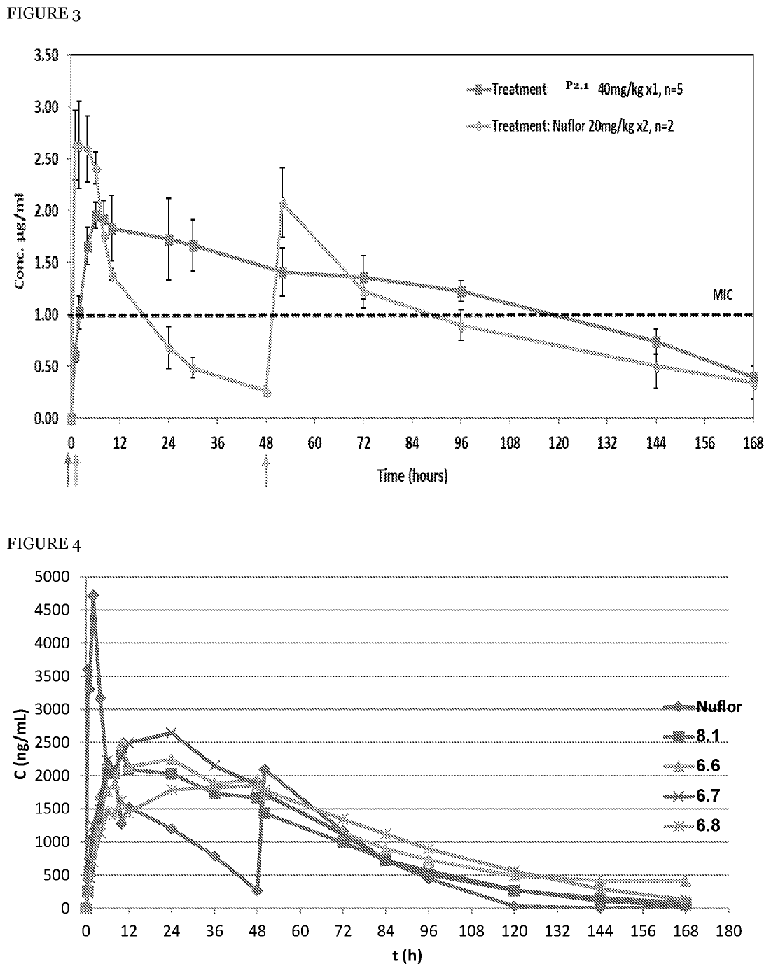

The results for the plot of plasma concentrations against time for the parent florfenicol compound are presented in the FIG. 3, for relevant comparisons. The dashed line on each graph indicates the likely maximum MIC90 for typical swine respiratory disease target pathogens. The error bars indicate the standard error of the mean. The arrows indicate the administration times. The diamonds (♦) represent Nuflor, designated as “Treatment: Nuflor 20 mg/kg×2, n=2”), and solid squares (▪) preparation 2.1 (designated as “Treatment P2.1, 40 mg/kg xl, n=5”), with “Conc. (μg/ml)” indicating the blood plasma concentration of florfenicol, and “Time (hours)” indicating time elapsed from the beginning of the experiment, in hours.

The pharmacokinetic parameters that were obtained for these data are summarized in Table 5 below.

TABLE 5

| Parameter | P2.1 40 mg/kg | Nuflor 20 mg/kg × 2 |
|---|---|---|
| Terminal t_{1/2} (h) | 43.5 (36.7-53.2) | 53.1 h (20.3-78.2) |
| AUC_{inf} (μg × h × mL⁻¹) | 224.9 | 176.0 |
| AUC over MIC (AUIC) | 178.7 | 84.4 |
| (μg × h × mL⁻¹) | | |
| Time percentile over MIC (%) | 79.4 | 47.9 |
| Cₘₐₓ (μg/mL) | 2.24 (1.62-2.79) | 2.77 (2.28-3.25) |
| Tₘₐₓ (h) | 10.8 (6-24) | 8 (6-10) |

The terminal half-life of Nuflor® was calculated from the second injection; the data from the first injection show significantly shorter half-life indicative of rapid elimination in the early stages. The maximal concentration reported for Nuflor arm is the maximal concentration of the first injection.

It can be readily seen that the preparation according to the invention produces higher relevant exposure to florfenicol, as demonstrated by the AUIC and the time percentile over MIC, after a single injection, relatively to the commercial product.

### Example 6

To evaluate the capability of the system to handle ultra-high loading of drugs, the following formulations of florfenicol were also prepared along the lines described herein. Preparation 6.1 contained about 33 wt % of florfenicol, 6.2 about 36 wt %, and 6.3 about 39 wt %.

The compositions were syringeable via the 16 G needle, injectable thereafter, and showed reverse thermal behavior, e.g. gelled at heating and liquefied again upon cooling. The release profiles and rheology data are summarized in the Table 6 below.

TABLE 6

| | Preparation 6.1 | | Preparation 6.2 | | Preparation 6.3 | |
|---|---|---|---|---|---|---|
| | Wt (g) | % w/w | Wt (g) | % w/w | Wt (g) | % w/w |
| Florfenicol | 8.0 | 32.6 | 8.5 | 36.2 | 9.25 | 39.3 |
| Poloxamer 407 | 3.0 | 12.2 | 3.0 | 12.8 | 2.5 | 10.6 |
| Klucel ® EF | 0.2 | 0.8 | 0.2 | 0.8 | 0.2 | 0.8 |
| DDW | 11.4 | 46.2 | 10.1 | 43.3 | 10.0 | 42.6 |
| NMP | 2 | 8.2 | 1.6 | 6.9 | 1.6 | 6.9 |
| Time(hr) | mean | sd | mean | sd | mean | sd |
| 0.25 | 1.41 | 0.31 | 1.59 | 0.40 | 2.26 | 0.47 |
| 0.5 | 2.41 | 0.35 | 2.85 | 0.63 | 4.28 | 0.93 |
| 1 | 4.38 | 0.87 | 5.41 | 1.09 | 7.69 | 1.17 |
| 1.5 | 5.94 | 1.32 | 7.42 | 1.57 | 11.33 | 1.54 |
| 2 | 7.19 | 1.72 | 9.36 | 2.07 | 14.64 | 1.32 |
| 3 | 9.33 | 2.36 | 13.14 | 3.10 | 20.54 | 1.27 |
| 4 | 11.09 | 2.71 | 16.40 | 4.21 | 25.56 | 1.42 |
| 5 | 12.57 | 3.22 | 19.10 | 5.10 | 29.44 | 1.77 |
| 6 | 14.27 | 3.55 | 21.04 | 5.31 | 33.14 | 1.31 |
| 7 | 15.64 | 3.55 | 23.39 | 6.01 | 36.91 | 1.64 |
| 8 | 17.12 | 4.00 | 25.23 | 6.10 | 40.65 | 0.84 |
| 24 | 36.45 | 7.16 | 59.74 | 7.54 | 77.95 | 5.15 |

Rheology

| | | | |
|---|---|---|---|
| Max viscosity | 13.1 Pa*s | 34.2 Pa*s | 18.9 Pa*s |
| Min viscosity | 0.28 Pa*s | 2.3 Pa*s | 1.01 Pa*s |
| Gelation t° C. | 24.4° C. | 18.2° C. | 26.2° C. |
| Gelation range | 23.9-28.4 | 18.0-20.3 | 25.8-28.1 |

It can be readily seen that the formulations created gels responsive to temperature increase, released the drug in a controlled manner with a low variability, as evidenced by low relative standard deviation at each point.

Further compositions were prepared at 45 wt % loading and higher. Florfenicol was sieved through 50-micron mesh, to obtain lower-particle size fraction. The formulations and the results are summarized in the table 7 below.

TABLE 7

| | Preparation 6.4 | | Preparation 6.5 | | Preparation 6.6 | | Preparation 6.7 | | Preparation 6.8 | |
|---|---|---|---|---|---|---|---|---|---|---|
| | Wt (g) | % w/w | Wt (g) | % w/w | Wt (g) | % w/w | Wt (g) | % w/w | Wt (g) | % w/w |
| Florfenicol | 45.0 | 45.0 | — | — | — | — | — | — | 47.5 | 47.5 |
| Florfenicol | — | — | 45.0 | 45.0 | 45.0 | 45.0 | 47.5 | 47.5 | — | — |
| sieved | | | | | | | | | | |
| Poloxamer 407 | 12.0 | 12.0 | 12.0 | 12.0 | 10.0 | 10.0 | 9.0 | 9.0 | 9.0 | 9.0 |
| Klucel ® EF | 0.5 | 0.5 | 0.5 | 0.5 | 0.5 | 0.5 | 0.4 | 0.4 | 0.4 | 0.4 |
| DDW | 37.5 | 37.5 | 37.5 | 37.5 | 39.5 | 39.5 | 38.1 | 38.1 | 38.1 | 38.1 |
| NMP | 5.0 | 5.0 | 5.0 | 5.0 | 5.0 | 5.0 | 5.0 | 5.0 | 5.0 | 5.0 |
| Time(hr) | mean | sd | mean | sd | mean | sd | mean | sd | mean | sd |
| 0.5 | 21.76 | 3.91 | 17.64 | 4.24 | 10.51 | 2.38 | 6.96 | 1.72 | 10.03 | 2.29 |
| 1 | 26.14 | 5.38 | 22.02 | 5.75 | 13.35 | 2.50 | 9.40 | 1.66 | 12.35 | 2.29 |
| 2 | 30.91 | 5.40 | 27.45 | 6.10 | 18.28 | 2.67 | 13.72 | 1.52 | 16.42 | 2.23 |
| 4 | 39.71 | 5.53 | 35.80 | 6.28 | 27.11 | 2.88 | 21.74 | 1.27 | 24.02 | 2.07 |
| 6 | 47.61 | 5.43 | 43.25 | 6.05 | 35.31 | 3.04 | 29.12 | 1.23 | 31.11 | 2.11 |
| 24 | 86.47 | 5.00 | 77.75 | 3.87 | 76.36 | 3.09 | 70.49 | 1.89 | 68.40 | 3.21 |
| 48 | 99.9 | 1.38 | 90.8 | 1.07 | 95.68 | 2.37 | 91.77 | 2.11 | 84.41 | 1.38 |

Rheology

| | | | | | |
|---|---|---|---|---|---|
| Min viscosity | NP | NP | 0.95 Pa*s | 0.75 Pa*s | 0.57 Pa*s |
| Max viscosity | NP | NP | 16.8 Pa*s | 12.9 Pa*s | NP |
| Gelation t° C. | 16.7° C. | 17.1° C. | 21.5° C. | 24.2 | NP |

The dissolution testing was carried out using paddle over disk method. The amount of ca. 1 g was tested in 900 mL of USP phosphate buffer pH 6.8, with 1% of CTAB added. Rheometry was performed at 500 reciprocal seconds with gap of 500 μm.

It can be seen from the results that smaller particle size does not adversely affect the release profiles, at very high loading perhaps slightly accelerating the drug release, and that ultra-high loading of florfenicol can be obtained as an injectable formulation.

### Example 7

Further compositions were prepared at 47.5 wt % loading. Sieved florfenicol was used, as in the Example 6. The formulations and the results are summarized in the table 8 below.

TABLE 8

| | Preparation 7.1 | | Preparation 7.2 | | Preparation 7.3 | | Preparation 7.4 | |
|---|---|---|---|---|---|---|---|---|
| | Wt (g) | % w/w | Wt (g) | % w/w | Wt (g) | % w/w | Wt (g) | % w/w |
| Florfenicol* | 57 | 47.5 | 57 | 47.5 | 57 | 47.5 | 57 | 47.5 |
| Poloxamer 407 | 10.2 | 8.5 | 10.2 | 8.5 | 10.2 | 8.5 | 10.8 | 9 |
| Klucel ® EF | 0.12 | 0.1 | — | — | 0.12 | 0.1 | 0.12 | 0.1 |
| DDW | 46.68 | 38.9 | 46.8 | 39 | 40.68 | 33.9 | 46.08 | 38.4 |
| NMP | 6 | 5 | 6 | 5 | 12 | 10 | 6 | 5 |
| Time(hr) | mean | sd | mean | sd | mean | sd | mean | sd |
| 0.5 | 8.97 | 3.77 | 24.31 | 8.04 | 7.16 | 2.42 | 24.56 | 2.94 |
| 1 | 11.15 | 3.98 | 26.80 | 7.80 | 9.72 | 2.50 | 27.15 | 3.53 |
| 2 | 15.40 | 4.49 | 30.98 | 7.35 | 14.14 | 2.38 | 31.38 | 4.14 |
| 4 | 23.36 | 5.31 | 38.21 | 6.81 | 22.32 | 2.18 | 38.19 | 4.16 |
| 6 | 30.48 | 6.02 | 44.52 | 6.60 | 29.72 | 1.91 | 44.27 | 4.26 |
| 24 | 69.82 | 7.55 | 77.28 | 4.67 | 71.41 | 0.99 | 77.95 | 4.42 |
| 48 | 90.04 | 4.27 | 94.71 | 3.18 | 92.76 | 2.09 | 96.84 | 1.65 |

Rheology

| | | | | |
|---|---|---|---|---|
| Min viscosity | 0.34 Pa*s | 0.34 Pa*s | 0.71 Pa*s | 0.38 Pa*s |
| Max viscosity | 10.05 Pa*s | 9.95 Pa*s | 7.9 Pa*s | 12.7 Pa*s |
| Gelation t° C. | 27.23° C. | 29.86° C. | 28.09° C. | 27.23° C. |

*sieved florfenicol

It can be readily seen from the results that the compositions comprising 47.5 weight percent of florfenicol can be made injectable, e.g. with good viscosity at ambience and suitable gelation point.

Additionally, it can be seen that even with low amount of hydroxypropyl cellulose (see, e.g. preparation 7.1 vs. 6.7) the release profile remains stable, with relatively low RSD (although indeed the variability is slightly higher with 7.1).

Quite unexpectedly, the variability without hydroxypropyl cellulose (preparation 7.2) was still within the pharmaceutically acceptable range, although even as little as 0.1% of cellulose additive reduces the variability significantly, without adversely affecting the release profile. Moreover, adding more co-solvent (preparation 7.3 vs. 7.1) improves further the variability, and even more so versus preparation 7.2 with no cellulose additive.

### Example 8

To further demonstrate the effect of the invention in vivo, another pharmacokinetic study was performed to demonstrate prolonged and effective plasma levels from a single administration of florfenicol in pigs.

A total of 20 pigs received in parallel either 40 mg/kg of the one-shot treatment of the preparations 6.6-6.8, or 30 mg/kg of Nuflor® (Merck Animal Health—florfenicol 30% solution in NMP), administered according to the manufacturer's recommendations. Additionally, a preparation (designated herein as 8.1) comprising 40 wt % of florfenicol, 12 wt % of poloxamer 407, 0.5 wt % of Klucel EF, 5 wt % of NMP and 42.5 wt % of water, with the gelation point of 21.7° C., was administered at 40 mg/kg. The release profile of the preparation 8.1 at the same conditions as in the Example 7 is demonstrated in the table 9 below.

TABLE 9

| Time(hr) | 0 | 0.5 | 1 | 2 | 4 | 6 | 24 | 48 |
|---|---|---|---|---|---|---|---|---|
| mean | 0 | 18.85 | 23.01 | 26.70 | 32.97 | 38.48 | 64.37 | 72.78 |
| RSD | 0 | 5.98 | 6.74 | 6.51 | 6.20 | 5.78 | 3.05 | 1.82 |

Blood samples were taken at time points 0, 0.5, 1, 2, 4, 6, 8, 10, 12, 24, 36, 48, 50, 72, 84, 96, 120, 144, and 168 hours.

The plot of blood plasma concentrations of florfenicol versus time is demonstrated in FIG. 4. In FIG. 4, the blood plasma concentrations of florfenicol at every sample point are demonstrated. The diamonds (♦) represent Nuflor, solid squares (▪) preparation 8.1, solid triangles (▴) preparation 6.6, and X-signs (x) preparation 6.7, and the asterisks (*) preparation 6.8, with “C (ng/mL)” indicating the blood plasma concentration of florfenicol, and “t (h)” indicated time elapsed from the beginning of the experiment, in hours.

It can be readily seen from the results that the commercially available product is rapidly eliminated from the blood of pigs, whereas all the preparations according to the invention maintain blood plasma levels above 1000 ng/mL for between 72 to 84 hours on average. It is noteworthy that the dose-corrected AUC of the treatments is comparable between groups, indicating that bioavailability was not reduced by the controlled-release formulations. The peak plasma concentration was evidently highest in the immediate-release commercial product; however, preparation 6.7 exhibited significantly higher peak concentration than preparation 6.8, which was only different in the particle size of the drug.

The times above minimal inhibitory concentration of Streptococcus suis, a virulent swine pathogen (currently considered 2 mcg/mL), of the tested articles, are presented in the table 10 below.

TABLE 10

| Above MIC | Nuflor | P8.1 | P6.6 | P6.7 | P6.8 |
|---|---|---|---|---|---|
| Time (h) | 7.44 | 18.5 | 27.8 | 34.2 | 14.8 |

It is evident from the results that the tested preparations according to the invention give superior results with a significant clinical potential to combat S. suis.

### Example 9

To demonstrate the ability of the compositions according to the invention to release other antibiotics, formulations comprising 30 wt % of amoxicillin were prepared. Preparation 9.1 contained both the co-solvent and the cellulose derivative at least partially soluble in organic solvents (hydroxypropyl cellulose), preparation 9.2 only hydroxypropyl cellulose, and 9.3 none of the additional excipients. The formulations were prepared along the lines as described for florfenicol.

The compositions were syringeable via the 16 G needle, injectable thereafter, and showed reverse thermal behavior, e.g. gelled at heating and liquefied again upon cooling. The release profiles data are summarized in the Table 11 below.

TABLE 11

| | Preparation 9.1 | | Preparation 9.2 | | Preparation 9.3 | |
|---|---|---|---|---|---|---|
| | Wt (g) | % w/w | Wt (g) | % w/w | Wt (g) | % w/w |
| Amoxycillin | 6.0 | 30 | 6.0 | 30 | 6.0 | 30 |
| Poloxamer 407 | 2.4 | 12 | 2.4 | 12 | 2.4 | 12 |
| Klucel ® EF | 0.15 | 0.75 | 0.15 | 0.75 | — | — |
| DDW | 9.45 | 47.25 | 11.45 | 57.25 | 11.6 | 58 |
| NMP | 2.0 | 10 | — | — | — | — |
| Time(hr) | mean | sd | mean | sd | mean | sd |
| 0.5 | 2.99 | 0.94 | 2.52 | 0.45 | 3.14 | 0.50 |
| 1 | 6.34 | 1.40 | 5.10 | 0.58 | 5.61 | 0.47 |
| 1.5 | 9.26 | 1.65 | 7.69 | 0.55 | 7.83 | 0.47 |
| 2 | 11.76 | 1.81 | 10.04 | 0.75 | 9.68 | 0.66 |
| 3 | 16.68 | 2.76 | 14.12 | 1.17 | 12.63 | 1.18 |
| 4 | 20.90 | 3.43 | 19.03 | 1.63 | 16.06 | 1.88 |
| 5 | 25.39 | 3.16 | 22.94 | 2.27 | 21.59 | 6.45 |
| 6 | 30.12 | 5.44 | 26.56 | 3.10 | 21.40 | 3.31 |
| 7 | 34.14 | 6.85 | 29.14 | 2.93 | 23.40 | 2.60 |
| 8 | 36.52 | 4.86 | 33.34 | 5.34 | 25.65 | 5.81 |
| 24 | 68.70 | 7.66 | 84.69 | 22.44 | 77.00 | 21.35 |

It can be readily seen that the formulations created gels, released the drug in a controlled manner with a low variability, as evidenced by low RSD at each point, but without either NMP or Klucel the drug release at later stage becomes more erratic, which might indicate the formation of a less stable gel in absence of both excipients.

### Example 10

To further demonstrate the ability of the compositions according to the invention to release other antibiotics, formulations comprising 15 wt % of tylosin were prepared. Preparation 10.1 contained both the co-solvent and the cellulose derivative at least partially soluble in organic solvents (hydroxypropyl cellulose), preparation 10.2 only hydroxypropyl cellulose, and 10.3 none of the additional excipients. The formulations were prepared along the lines as described for florfenicol.

The compositions were syringeable via the 16 G needle, injectable thereafter, and showed reverse thermal behavior, e.g. gelled at heating and liquefied again upon cooling. The release profiles data are summarized in the Table 12 below.

TABLE 12

| | Preparation 9.1 | | Preparation 9.2 | | Preparation 9.3 | |
|---|---|---|---|---|---|---|
| | Wt (g) | % w/w | Wt (g) | % w/w | Wt (g) | % w/w |
| Tylosin | 3.0 | 15 | 3.0 | 15 | 3.0 | 15 |
| Poloxamer 407 | 2.92 | 14.6 | 2.4 | 12 | 2.4 | 12 |
| Klucel ® EF | 0.18 | 0.9 | 0.15 | 0.76 | — | — |
| DDW | 11.47 | 57.3 | 14.45 | 72.24 | 14.6 | 73 |
| NMP | 2.43 | 12.2 | — | — | — | — |
| Time(hr) | mean | sd | mean | sd | mean | sd |
| 0.5 | 11.40 | 1.80 | 5.48 | 0.09 | 4.83 | 1.65 |
| 1 | 20.95 | 1.16 | 9.33 | 0.99 | 8.24 | 2.96 |
| 1.5 | 35.71 | 1.47 | 12.51 | 1.56 | 11.26 | 4.24 |
| 2 | 37.27 | 1.54 | 15.63 | 1.75 | 14.35 | 5.54 |
| 3 | 46.75 | 1.72 | 21.51 | 2.09 | 20.05 | 7.60 |
| 4 | 52.50 | 1.28 | 25.64 | 2.04 | 25.18 | 9.34 |
| 5 | 57.97 | 1.95 | 31.04 | 1.60 | 30.63 | 11.46 |
| 6 | 61.14 | 1.07 | 36.23 | 2.67 | 36.67 | 13.35 |
| 7 | 70.25 | 3.08 | 40.72 | 4.02 | 41.42 | 14.09 |
| 8 | 72.71 | 3.49 | 45.10 | 6.39 | 46.19 | 15.39 |
| 24 | 94.05 | 3.02 | 97.94 | 1.60 | 96.85 | 2.08 |

It can be readily seen that the formulations created gels, and released the drug in a controlled manner. The preparation 10.1 had slightly more poloxamer to compensate the increased drug solubility with the NMP. The release profile of 10.1 demonstrates with a low variability, as evidenced by low RSD at each point, particularly in the intermediate times. Preparation 10.2 shows slightly more variability, but without both NMP and Klucel the drug release becomes more variable.

1. A pharmaceutical composition comprising a biologically active agent, poloxamer, an aqueous carrier, and an organic co-solvent, wherein said composition is an injectable composition at room temperature, wherein, if said active agent is at a concentration below 35 wt % the pharmaceutical composition, then the pharmaceutical composition further comprises a cellulose-based material which is at least partially soluble in organic solvents.

2. The pharmaceutical composition according to claim 1, wherein a concentration of said biologically active agent is between 10 wt % and 35 wt %.

3. The pharmaceutical composition according to claim 1, wherein a concentration of said biologically active agent is above 35 wt %, and wherein said composition is devoid of a cellulose-based material which is at least partially soluble in organic solvents.

4. The pharmaceutical composition according to claim 1, wherein a concentration of said biologically active agent is above 35 wt %, and wherein said composition further comprises a cellulose-based material which is at least partially soluble in organic solvents.

5. The pharmaceutical composition according to claim 3, wherein a concentration of said biologically active agent is between 35 wt % and 50 wt %.

6. The pharmaceutical composition of claim 1, wherein said biologically active agent is selected from florfenicol, lincomycin, tylosin, metronidazole, tilmicosin, spiramycin, erythromycin, tulathromycin, tiamulin, ampicillin, amoxicillin, clavulanic acid, penicillin, streptomycin, trimethoprim, sulfonamide, sulfamethoxazole, pleuromutilin, avilosin, tylvalosin, doxycycline, and oxytetracycline.

7. The pharmaceutical composition of claim 1, wherein said biologically active agent is florfenicol.

8. The pharmaceutical composition of claim 6, wherein said biologically active agent is present in said composition in a loading of between about 25 wt % to about 50 wt %.

9. The pharmaceutical composition of claim 1, wherein said organic co-solvent is present at an amount of between about 5 to about 15% wt.

10. The pharmaceutical composition of claim 1, wherein said cellulose-based material which is at least partially soluble in organic solvents is hydroxypropyl cellulose.

11. The pharmaceutical composition of claim 1, wherein said organic solvent is selected from the group consisting of N-methyl pyrrolidone (NMP), dimethyl sulfoxide (DMSO), PEG 400, propylene glycol, and ethanol.

12. The pharmaceutical composition of claim 1, wherein said organic solvent is N-methyl pyrrolidone.

13. The pharmaceutical composition of claim 1, wherein said organic solvent is N-methyl pyrrolidone, and wherein said cellulose-based material which is at least partially soluble in organic solvents is hydroxypropyl cellulose, and further wherein said biologically active agent is florfenicol at a concentration of at between 25 wt % and 50 wt %.

14. The pharmaceutical composition of claim 1, wherein said organic solvent is N-methyl pyrrolidone, and wherein said biologically active agent is florfenicol, and further wherein a concentration of said florfenicol is between 35 wt % and 50 wt %.

15. A pharmaceutical composition as defined in claim 1 for use in treating of a veterinary infection in a non-human animal by administering to said animal a pharmacologically effective dose of an antibiotic in said composition.

16. The pharmaceutical composition of claim 15, wherein said composition is administered once to said non-human animal per the course of treatment.

17. The pharmaceutical composition of claim 15, wherein said administration comprises intramuscular injection, or subcutaneous injection.

18. The pharmaceutical composition of claim 15, wherein said infection is caused by a swine pathogen.
